(19) United States
(12) Patent Application Publication
(10) Pub. No.: US 2023/0234931 A1
(43) Pub. Date: Jul. 27, 2023

(54) NON-STEROIDAL ANTI-INFLAMMATORY COMPOUNDS

(71) Applicant: University of Houston System, Houston, TX (US)

(72) Inventor: Ke-He RUAN, Bellaire, TX (US)

(21) Appl. No.: 18/158,670

(22) Filed: Jan. 24, 2023

Related U.S. Application Data
(60) Provisional application No. 63/302,782, filed on Jan. 25, 2022.
(60) Provisional application No. 63/306,737, filed on Feb. 4, 2022.

(51) Int. Cl. C07D 263/54; A61P 29/00

(52) U.S. Cl. CPC C07D 263/54; A61P 29/00

(57) ABSTRACT

The disclosure provides 2-amino 4-nitrophenol and 2,4-diaminophenol derivatives, which can be used as anti-inflammatory drugs, prostaglandin E₂ (PGE₂) synthesis and activity inhibitors, and microsomal PGE₂ synthase-1 (mPGES-1) inhibitors.

This invention claims benefit and priority to U.S. Provisional Application No. 63/302,782 filed on Jan. 25, 2022 and U.S. Provisional Application No. 63/306,737 filed on Feb. 4, 2022, which are both incorporated herein by reference in their entirety.

This invention was made with government support under HL56712 and HL79389 awarded by the National Institutes of Health. The government has certain rights in the invention.

### TECHNICAL FIELD

The present disclosure relates to non-steroidal anti-inflammatory compositions, and methods for their manufacture, to be used in the treatment of inflammation, inflammatory diseases or disorders, pain and/or fever. The disclosed compositions contain, as an active ingredient, derivatives of 2-amino 4-nitrophenol and 2,4-diaminophenol having modifications of the functional groups at the position 1, —OH and position 2, —NH using benzoic acid derivatives. Such derivatives are shown to function to inhibit the activities of prostaglandin E₂ (PGE₂) and microsomal PGE₂ synthase-1 (mPGES-1).

### BACKGROUND

Non-steroidal anti-inflammatory drugs (NSAIDs), including aspirin, Advil, Motrin, Celebrex, and others, are commonly used to effectively treat inflammation, pain, and fever through inhibition of cyclooxygenase-1 (COX-1) and/or -2 (COX-2). Their anti-inflammatory effects are a result of reducing pro-inflammatory prostaglandin E₂ (PGE₂), which is synthesized by the inducible COX-2 that is coupled to inducible microsomal PGE₂ synthase-1 (mPGES-1) (1-6). In the arachidonic acid (AA) metabolism pathway, COX-1 and -2 are upstream enzymes. They are also coupled to other downstream synthases to produce a variety of prostanoids, such as prostacyclin (PGI₂) synthase, which produces PGI₂ that is involved in vascular protection through anti-platelet aggregation and vasodilation (7-10), thromboxane A₂ (TXA₂) synthase, which produces TXA₂, an endogenous anti-bleeding factor, and non-inducible PGE₂ synthase, which produces the basal level PGE₂ involved in gastrointestinal (GI) protection (7, 11).

Accordingly, the major mechanism of the current COX-2 inhibitors used to treat inflammation and pain is through the reduction of the inflammatory PGE₂ production by inhibiting upstream COX-2 (4-7). However, inhibiting COX-2 also results in reduction of the production of other downstream enzyme-produced prostanoids, such as prostacyclin, which is a major cardiovascular protector. The NSAID inhibition of upstream COX could cause severe side effects, such as GI and cardiovascular insults from over-inhibition of PGI₂ and basal level PGE₂ as well as excessive bleeding from over-inhibition of TXA₂ biosynthesis (11). Current COX-2 inhibitors, such as Celebrex, increase heart disease risk by decreasing prostacyclin (PGI₂) which has been demonstrated in laboratories to clinical trials (21-22).

A solution to effectively reduce inflammatory PGE₂ production without reducing prostacyclin (PGI₂) through COX-2 inhibition is currently unavailable. Accordingly, novel anti-inflammatory compositions, without the inherent side effects of COX-2 inhibitors, are greatly needed.

### SUMMARY

The present disclosure relates to anti-inflammatory, pain and/or fever reducing compositions, and more particularly to anti-inflammatory compositions containing, as active ingredients, derivatives of 2-amino-4-nitrophenol and 2,4-diaminophenol (herein referred to as “derivative compounds”). Such derivative compounds include, for example, those having modification of the functional groups at the position 1, —OH and the position 2, —NH of 2-amino-4-nitrophenol and 2,4-diaminophenol using benzoic acid derivatives such as, for example, 4-(fluoro-, difluoro- or trifluoro-methyl)-benzoic acid; 4-(methyl-, dimethyl- or trimethyl)-benzoic acid; 4-(chloro-, dichloro, or trichloro-methyl)-benzoic acid, and 4-(phenyl, diphenyl- or tri-phenol)-benzoic acid to name a few.

The present disclosure provides the chemical structures of the derivative compounds as well as methods used for chemical modification and synthesis of the derivative compounds which can then be used as anti-inflammatory, pain, and/or fever reduction compounds. Said derivatives are demonstrated herein to inhibit the activities of PGE₂ and microsomal PGE₂ synthase-1 (mPGES-1) leading to a reduction in prostaglandin E₂ (PGE₂) biosynthesis.

Accordingly, the present disclosure provides compositions for inhibiting inflammation in a subject comprising administering to the subject, an effective amount of one or more of a derivative compound as described herein in a pharmaceutically acceptable form.

In an embodiment, use of the derivative compounds and compositions disclosed herein for treating a subject suffering from inflammation, or at risk for developing an inflammatory reaction is provided, the use comprising administering to the subject, an effective amount of one or more of the derivative compounds in a pharmaceutically acceptable form. For such treatments, the administration of the derivative compound(s) is used, through its inhibition of PGE₂ and mPGES-1 leading to a reduction in PGE₂ biosynthesis, to inhibit or reduce the symptoms of inflammation.

In an embodiment, use of the derivative compounds and compositions disclosed herein for treatment of a subject suffering from inflammation or having a risk factor for developing an inflammatory disease is provided, the method comprising administering to the subject, an effective amount of a derivative compound in a pharmaceutically acceptable form.

In further embodiments, pharmaceutical compositions comprising the derivative compounds and a pharmaceutical acceptable carrier are provided. The derivative compounds exhibit properties for use as therapeutic agents, e.g. in the treatment of inflammation and the symptoms associated with inflammation, e.g., pain and fever.

### BRIEF DESCRIPTION OF THE FIGURES

In order to better understand the subject matter that is disclosed herein and to exemplify how it may be carried out in practice, embodiments will now be described, by way of non-limiting example, with reference to the accompanying drawings. With specific reference to the drawings, it is stressed that the particulars shown are by way of example and for purposes of illustrative discussion of embodiments of the disclosure.

FIG. 1A-B. FIG. 1A. Binding of 1-F, 2-F and 3-F derivatives to mPGES-1 by docking study using the crystal 3D structure of the native trimer form of human mPGES-1 (PDB ID: 4YL3). FIG. 1A Chemical structure of 1F, 2F and 3F derivatives. FIG. 1B The binding of 1-F, 2-F and 3-F into the pocket of the trimer of mPGES-1.

FIG. 2A-B Binding of 1-Me, 2-Me and 3-Me derivatives to mPGES-1 by docking study using the crystal 3D structure of the native trimer form of human mPGES-1 as described in method section. FIG. 2A Chemical structure of 1-Me, 2-Me and 3-Me derivatives. FIG. 2B The binding of 1Me, 2Me and 3Me into the pocket of the trimer of mPGES-1.

FIG. 3A-D. Binding of 1-Cl, 2-Cl and 3-Cl, and 1-phenyl, 2-phenyl and 3-phenyl derivatives to mPGES-1 by docking studies using the crystal 3D-structure of the native trimer form of human mPGES-1 (PDB ID: 4YL3). FIG. 3A Chemical structure of 1-Cl, 2-Cl and 3-Cl derivatives. FIG. 3B Chemical structure of 1-phenyl, 2-phenyl and 3-phenyl derivatives. FIG. 3C The binding of Cl-derivatives into the pocket of the trimer of mPGES-1. FIG. 3D The binding of the phenyl-derivatives into the pocket of the trimer of mPGES-1.

FIG. 4A-B. FIG. 4A. Chemical synthesis of 3-F derivatives. The purification procedures are described in the examples section below. FIG. 4B Elution profile of normal-phase HPLC purification.

FIG. 5A-B. Structure determination of 2-[4-(trifluoromethyl) phenyl]-1,3-benzoxazol-5-amine. (FIG. 5A) ¹H-NMR of 2-[4-(trifluoromethyl) phenyl]-1,3-benzoxazol-5-amine taken in CDCl3 at 400 MHz. 3.76 (1H, s), 6.73-6.76 (1H, dd, J=8.4 and 2.4 Hz), 7.05 (1H, d, J=2.4 Hz), 7.36-7.38 (1H, d, J=8.8 Hz), 7.75-7.77 (2H, d, J=8.8 Hz), 8.31-8.33 (2H, d, J=8 Hz). (FIG. 5A) LC/MS spectrum of 2-[4-(trifluoromethyl)phenyl]-1,3-benzoxazol-5-amine. MH+279.1.

FIG. 6A-B. Chemical synthesis and structure determination of 2,4-diaminophenol 3-Me derivative. (FIG. 6A) chemical reaction step. (FIG. 6B) ¹H-NMR of 2-[4-(trimethyl) phenyl]-1,3-benzoxazol-5-amine taken in CDCl3 at 600 MHz. ¹H NMR: δ 1.28 (9H, s), 6.56 (1H, dd, J=8.4, 1.8 Hz), 7.27-7.44 (3H, 7.33 (ddd, J=8.0, 1.3, 0.4 Hz), 7.39 (dd, J=1.8, 0.5 Hz)), 7.63 (1H, dd, J=8.4, 0.5 Hz), 7.94 (2H, ddd, J=8.0, 1.6, 0.4 Hz).

FIG. 7. Chemical synthesis of 1-Cl derivative.

FIG. 8. Chemical synthesis of 1-Phe derivative.

FIG. 9. Dose response curves for the effects of the synthesized derivatives on mPGES-1-synthesized inflammatory PGE₂, compared with COX-2 inhibitor. HEK-293 cells expressing inflammatory PGE-producing Enzymelink, COX-2-10aa-mPGES-1 described previously (11) were treated with increasing concentrations (1 μM, 3 μM, and 10 μM), of the derivatives. Arachidonic acid (0.5 μM) was added to initiate the biosynthesis of PGE₂ through COX-2 and mPGES-1 catalysis (11). The produced PGE₂ was detected by ELISA (11). COX-2 inhibitor, NS-398 was used as positive control. An unrelated chemical compound (#6) was used as a negative control.

FIG. 10A-C. Anti-inflammatory and anti-cancer activities in cell cultural conditions. PC-3 migration assay treated with the lead compound #10. FIG. 10A The representative images of the PC-3 migration at 0 hour (left) and 39 hours (right) with DMSO, NS-398, and #10. FIG. 10B The time-course gap size of the PC-3 migration. The gap size was obtained using NIS-element software (NIKON). n=6. FIG. 10C Quantitative and significant analyses.

### DETAILED DESCRIPTION

The present invention relates to anti-inflammatory, pain and fever reducing compositions, and more particularly to anti-inflammatory compositions containing, as active ingredients, derivatives of 2-amino-4-nitrophenol and 2,4-diaminophenol (herein referred to as “derivative compounds”). Such derivative compounds include, for example, those having modification of the functional groups at the position 1, —OH and the position 2, —NH of 2-amino-4-nitrophenol and 2,4-diaminophenol using benzoic acid derivatives such as, for example, 4-(fluoro-, difluoro- or trifluoro-methyl)-benzoic acid; 4-(methyl-, dimethyl- or trimethyl)-benzoic acid; 4-(chloro-, dichloro, or trichloro-methyl)-benzoic acid, and 4-(phenyl, diphenyl- or tri-phenol)-benzoic acid to name a few.

In one embodiment, a derivative compound is one resulting from the modification of the position 1, —OH group (R1) and position 2 —NH group (R2) of 2-amino, 4-nitrol phenol resulting in products with anti-inflammatory properties and which are capable of inhibiting PGE₂ biosynthesis and activity, and mPGES-1 activity. The result is the compound of formula (I):

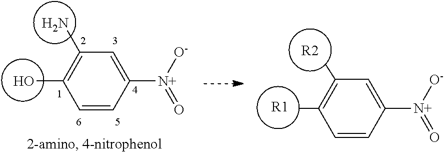

Compounds further include those where R1 and R2 are modified by benzoic acid resulting in products with anti-inflammatory properties and which are capable of inhibiting PGE₂ biosynthesis and activity, and mPGES-1 activity. The result is the compound of formula (III):

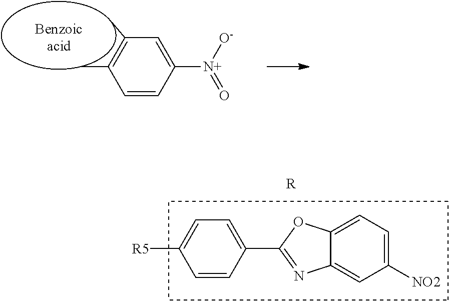

In an embodiment, derivative compounds include those where the R5 is a fluoro-, difluoro- or trifluoro-group and wherein said derivative compounds exhibit anti-inflammatory activity and/or inhibit PGE₂ biosynthesis and activity, and mPGES-1 activity:

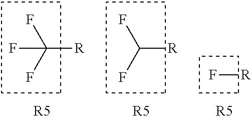

In another embodiment, derivative compounds include those where the R5 is a methyl-, dimethyl- or trimethyl-group and wherein said derivative compounds exhibit anti-inflammatory activity and/or inhibit PGE₂ biosynthesis and activity, and mPGES-1 activity:

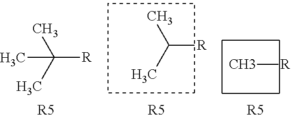

In another embodiment, derivative compounds include those where the R5 is a chloro-, dichloro, or trichloro-group and wherein said derivative compounds exhibit anti-inflammatory activity and/or inhibit PGE₂ biosynthesis and activity, and mPGES-1 activity:

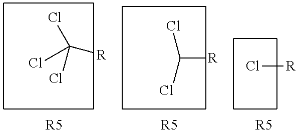

In another embodiment, derivative compounds include those where the R5 of is a phenyl, diphenyl- or triphenyl-group and wherein said derivative compounds exhibit anti-inflammatory activity and/or inhibit PGE₂ biosynthesis and activity, and mPGES-1 activity:

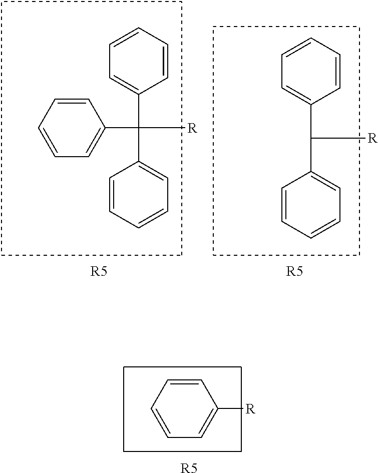

In another embodiment, derivative compounds include those resulting from the modification of the position 1, —OH group (R1) and position 2 —NH group (R2) of 2,4-diaminophenol resulting in products with anti-inflammatory properties and which are capable of inhibiting PGE₂ biosynthesis and activity, and mPGES-1 activity. Compound of formula (II):

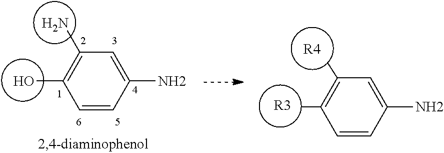

In another embodiment, derivative compounds include those where R3 and R4 are modified by benzoic acid resulting in products with anti-inflammatory properties and which are capable of inhibiting PGE₂ activity and biosynthesis, and mPGES-1 activity. Compound of formula (IV):

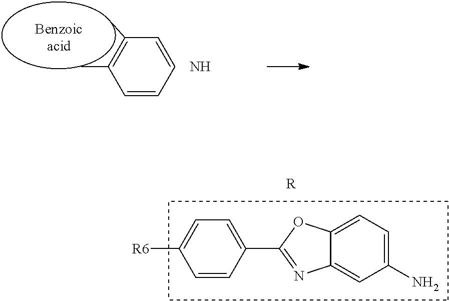

In an embodiment, derivative compounds are provided where the R6 is a fluoro-, difluoro- or trifluoro-group and wherein said derivative compounds exhibit anti-inflammatory activity and/or inhibit PGE₂ biosynthesis and activity, and mPGES-1 activity:

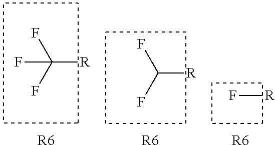

In another embodiment, provided derivative compounds include those where the R6 is a methyl-, dimethyl- or trimethyl-group and wherein said derivative compounds exhibit anti-inflammatory activity and/or inhibit PGE₂ biosynthesis and activity, and mPGES-1 activity:

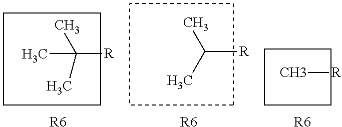

In another embodiment, provided derivative compounds include those where the R6 of the compound is a chloro-, dichloro-, or trichloro-group and wherein said derivative compounds exhibit anti-inflammatory activity and/or inhibit PGE₂ biosynthesis and activity, and mPGES-1 activity:

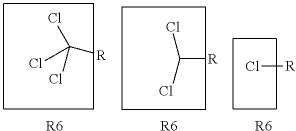

In another embodiment, provided derivative compounds include those where the R6 is a phenyl, diphenyl- or triphenyl-group and wherein said derivative compounds exhibit anti-inflammatory activity and/or inhibit PGE₂ biosynthesis and activity, and mPGES-1 activity:

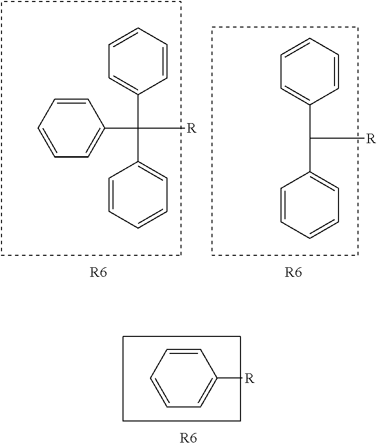

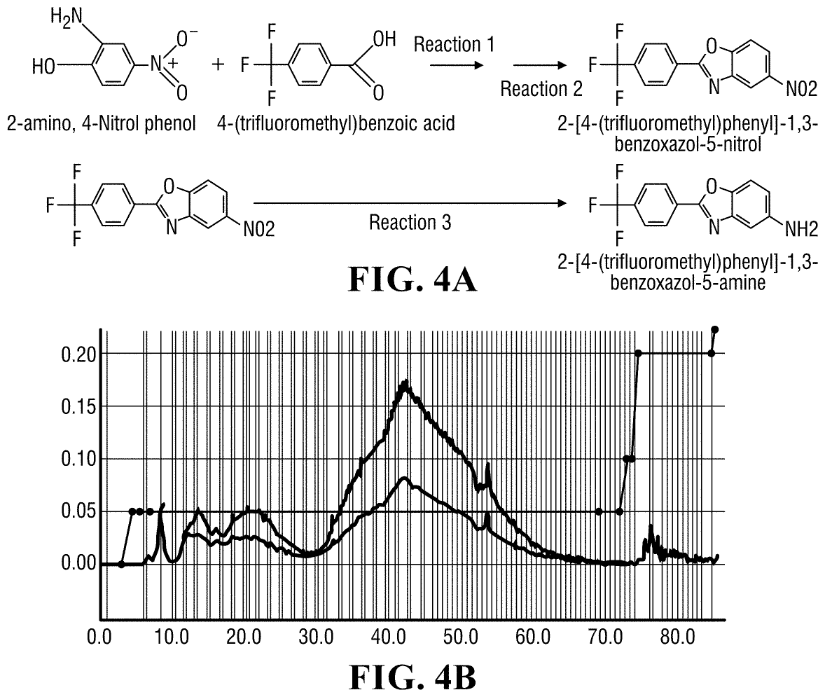

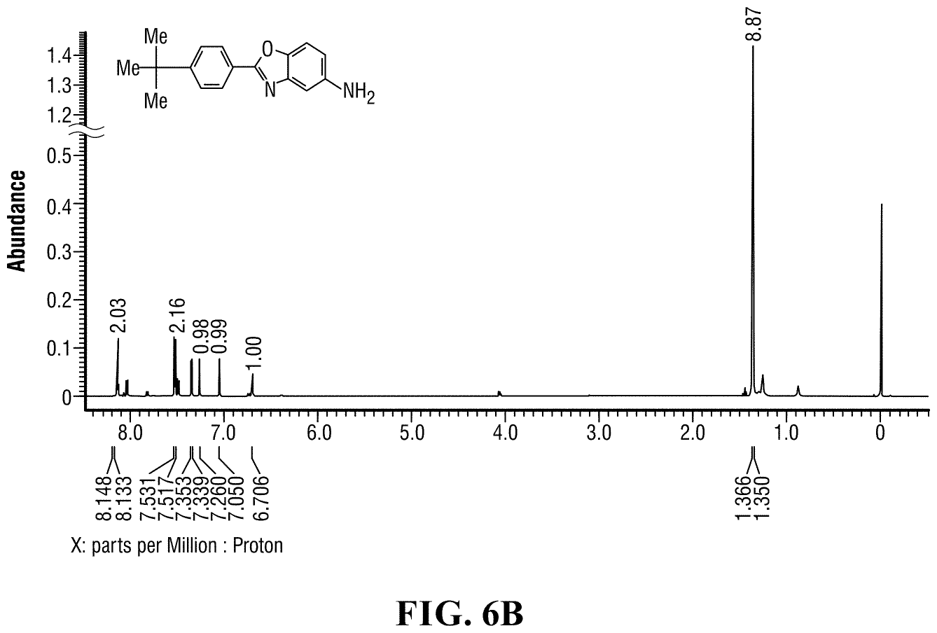

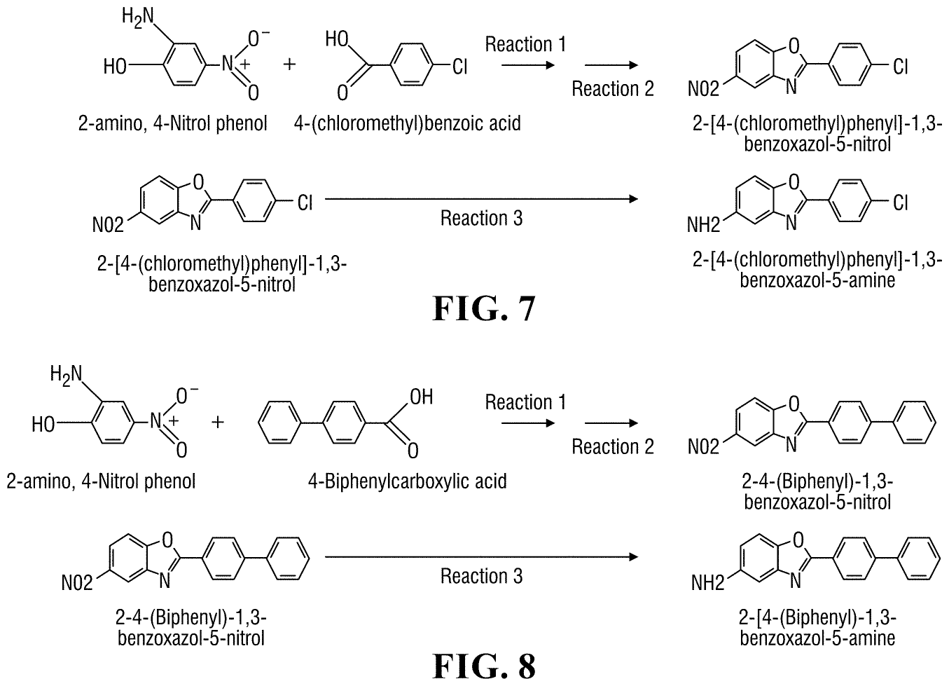

Methods for the synthesis of derivative compounds having anti-inflammatory activity are provided herein. In this regard, reference is made to FIG. 4, FIG. 6, FIG. 7 and FIG. 8, as well as the example section below for methods of producing the derivative compounds for use in treating inflammation and for inhibiting the activity of PGE₂ and mPGES-1.

In an embodiment, a method is provided wherein derivatives of 2-amino 4-nitrophenol are derived using a method comprising a first step of mixing a benzoic acid derivative with 2-amino, 4-nitrophenol under conditions wherein the benzoic acid reacts with the 2-amino, 4-nitrophenol to form a derivative compound. In a second step of the method, the resulting reaction mixture may then be neutralized to a pH of about 7.0. In yet a further third step, the synthesis of derivatives of 2,4-diaminophenol is provided wherein the product derivatives of 2-amino 4-nitrophenol obtain in the first and second step are further reacted to convert —NO₂ of the derivatives of the 2-amino 4-nitrophenol to —NH2 of the derivatives of 2,4-diaminophenol as final products.

Benzoic derivatives that may be used in the disclosed methods include, but are limited to, for example, 4-(fluoro-, difluoro- or trifluoro-methyl)-benzoic acid; 4-(methyl-, dimethyl- or trimethyl)-benzoic acid; 4-(chloro-, dichloro-, or trichloro-methyl)-benzoic acid, and 4-(phenyl, diphenyl- or tri-phenol)-benzoic acid to name a few.

In a specific, non-limiting embodiment, a method is provided for the synthesis of derivatives of 2-amino 4-nitrophenol is provided comprising the following steps: (Step 1): Polyphosphoric acid (PPA, Sigma-Aldrich) was first heated to 110° C. and 0.001-1 mol 2-amino-4 nitrophenol (Sigma-Aldrich) and 0.0015-1.5 mol (mol ratio of 1:1-1.5) corresponding benzoic acid (4-trifluoromethyl benzoic acid, Oakwood Chemical and 4-biphenylcarboxylic acid, Acros organics) were simultaneously added. The resulting mixture is then heated to 120-180° C. for 2-4 hours; (Step 2): At the end of the reaction, the solution is poured into ice-water and neutralized to pH 7.0. The precipitate is then filtered and collected as crude product.

In a non-limiting embodiment of the invention a method for the synthesis of the derivatives of 2,4-diaminophenol is provided comprising the additional (Step 3) wherein the product derivatives of 2-amino, 4-nitrophenol as described above are further reacted using Step 3: The crude mid product was obtained by recrystallizing via boiling in ethanol. The crude mid product is heated in 20 ml ethanol with Tin (II) chloride (SnCl₂) at 70° C. for 10-16 hours. After the reaction, the mixture is cooled to room temperature and poured into ice-water. Saturated Sodium bicarbonate (NaHCO₃) is then used for neutralizing the mixture. Using an alternative step 3, the crude mid-product and 10% palladium on activated charcoal is first dissolved in methanol. Then the mixture is bubbled with hydrogen gas at room temperature for 2 hours to acquire the crude final product:

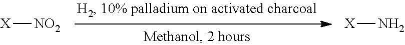

This crude final product was then filtered to obtain the aqueous layer. Finally, the aqueous layer obtained from step 3, or alternative step 3, was extracted twice with EtOAc (500 ml). The final combined organic layers were dried by anhydrous MgSO₄ and evaporated to obtain the final product (26,27).

Regarding the specific steps describe above, for production of the derivative compounds, it is understood that one skilled in the art may alter the conditions, e.g., temperatures and reaction times as well as the choice and concentrations of reagents while retaining the ability to successfully obtaining the derivative compounds of interest.

Provided herein is also a method of producing the derivative compounds in a form suitable for administration in vivo, the method comprising (a) obtaining the compounds as described herein according to various embodiments, and (b) formulating the compounds with at least one pharmaceutically acceptable carrier, whereby a preparation of the derivative compound is formulated for administration in vivo.

In a further aspect, certain embodiments provide pharmaceutical compositions comprising one or more of the derivative compounds as described herein, e.g., for use in any of the therapeutic methods for use in treating inflammation, inflammatory diseases or disorders, pain, and/or fever and inhibiting the activity of PGE₂ and mPGES-1. In one embodiment, pharmaceutical compositions comprising one or more derivative compounds and a pharmaceutically acceptable carrier are provided herein. Such compositions may be used for treatment of inflammatory diseases pain and/or fever. In another embodiment, a pharmaceutical composition comprises any of the one or more derivative compounds disclosed herein and at least one additional therapeutic agent, typically used for treatment of inflammatory disease.

Pharmaceutical compositions provided herein comprise a therapeutically effective amount of one or more of the derivative compounds dissolved or dispersed in a pharmaceutically acceptable carrier. The preparation of such pharmaceutical compositions containing at least one or more of the derivative compounds, and optionally an additional active ingredient, will be known to those of skill in the art in light of the present disclosure, as exemplified by Remington's Pharmaceutical Sciences, 18th Ed. Mack Printing Company, 1990. For human administration, it will be understood that preparations should meet sterility, pyrogenicity, general safety and purity standards as required by FDA Office of Biological Standards or corresponding authorities in other countries. Preferred compositions are lyophilized formulations or aqueous solutions.

As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it needs to be sterile for such routes of administration as injection. The derivative compounds described herein of certain embodiments (and any additional therapeutic agent) can be administered by any method or any combination of methods as would be known to one of ordinary skill in the art (see, for example, Remington's Pharmaceutical Sciences, 18th Ed. Mack Printing Company, 1990, incorporated herein by reference). Parenteral administration, in particular intravenous injection, may be used for administering of the compounds. Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Parenteral compositions include those designed for administration by injection, e.g. subcutaneous, intradermal, intra-lesional, intravenous, intra-arterial, intramuscular, intrathecal or intraperitoneal injection. For injection, the compounds may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the compounds may be in powder form for constitution with a suitable vehicle, e.g., sterile pyrogen-free water, before use. Sterile injectable solutions are prepared by incorporating the compounds in the required amount in the appropriate solvent with various other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi.

Pharmaceutical compositions comprising the derivative compounds may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The derivative compounds may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The pharmaceutical preparation of certain embodiments is a liquid composition, e.g., an aqueous solution. For injection purposes, the use of pure water as solvent is preferred. Other solvents which are suitable and conventional for pharmaceutical preparations can, however, also be employed. In a preferred embodiment, the pharmaceutical compositions are isotonic solutions. Further, there is no need for reconstitution at any stage of the preparation of the liquid solution formulation of these embodiments. The solution is a ready-to-use formulation.

Any of the derivative compounds, provided herein may be used in therapeutic methods described herein. For use in the therapeutic methods described herein, the compounds would be formulated, dosed, and administered in a fashion consistent with good medical practice. Factors for consideration in this context include the particular disorder being treated, the particular subject being treated, the clinical condition of the subject, the cause of the disease or condition, the site of delivery of the agent, the method of administration, the scheduling of administration, and other factors known to medical practitioners or those of skill in the art.

The present disclosure relates to compositions for prevention and/or treatment of inflammatory diseases or disorders and/or the symptoms associated with inflammation, e.g. pain and fever. In one embodiment, such treatments are designed to reduce the expression and/or activity of PGE₂ and/or mPGES-1 in the subject to be treated.

Since the compositions of the present disclosure contain the derivative compounds or a salt thereof as an active ingredient, it can inhibit inflammatory responses even at an early stage and exhibit a potent anti-inflammatory effect by regulating the expression and/or activity of PGE₂ and/or mPGES-1.

Specifically, the derivative compounds and compositions of the present disclosure can inhibit an early stage of inflammatory response by inhibiting the expression and/or activity of PGE₂ and/or mPGES-1. Based on the anti-inflammatory effect of the derivative compounds described herein, the compositions of the present invention may be compositions for preventing, ameliorating or treating inflammatory disease.

Such inflammatory disease is not limited in the kind thereof but may be selected from the group consisting of inflammatory lung disease, inflammatory liver disease, inflammatory bowel disease, autoinflammatory disease, inflammatory central nervous system disease, inflammatory skin disease, and allergic inflammatory disease. More specifically, the inflammatory disease may be selected from the group consisting of interstitial lung disease (ILD), non-alcoholic steatohepatitis (NASH), Crohn's disease, ulcerative colitis, rheumatoid arthritis, type 1 diabetes, lupus, multiple sclerosis, Parkinson's disease, scleroderma and psoriasis.

For the treatment of, for example, inflammation, pain and/or fever, the pharmaceutical form of the compositions comprising the derivative compounds (when used alone or in combination with one or more other additional therapeutic agents) will depend on the type of disease to be treated, the route of administration, the body weight of the patient, the severity and course of the disease, whether the compound is administered for preventive or therapeutic purposes, previous or concurrent therapeutic interventions, the patient's clinical history and response to the compound, and the discretion of the attending physician.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Example

### Materials and Methods

### General Method of Synthesis of 2,4-Diaminophenol Derivatives

Step 1: Polyphosphoric acid (PPA, Sigma-Aldrich) was first heated to 110° C. and 0.001-1 mol 2-amino-4 nitrophenol (Sigma-Aldrich) and 0.0015-1.5 mol (mol ratio of 1:1-1.5) corresponding benzoic acid (4-trifluoromethyl benzoic acid, Oakwood Chemical and 4-biphenylcarboxylic acid, Acros organics) were simultaneously added. Then the result mixture was heated to 120-180° C. for 2-4 hours. Step 2: At the end of the reaction, the solution was poured into ice-water and neutralized to pH7.0. The precipitate was filtered and collected as crude product. Step 3: Then the crude mid product was obtained by recrystallizing via boiling in ethanol. The crude mid product was heated in 20 ml ethanol with Tin (II) chloride (SnCl₂) at 70° C. for 10-16 hours. After the reaction, the mixture was cooling to room temperature and poured into ice-water. Saturated Sodium bicarbonate (NaHCO₃) was used for neutralized the mixture. Then the aqueous layer was extracted twice with EtOAc (500 ml). The final combined organic layers were dried by anhydrous MgSO₄ and evaporated to get the final product (26,27).

### Molecular Docking Studies

Molecular docking studies were performed by Molecular Operating Environment (MOE) software. To identity potential inhibitors of mPGES-1, the trimeric crystal structure of mPGES-1 (PDBID: 4YL3, 28) was downloaded from the Protein Data Bank and prepared by structure preparation. During the preparation, all the water molecular were removed and hydrogen atoms were added. All the ligands were from the previously virtual screening result library (11) and performed energy minimization. The docking process was based on general Docking method of MOE.

### Biological Activity Studies

Cell culture. HEK 293 and PC-3 cell lines were purchased from ATCC (VA, USA). HEK293 cells and HEK293 transfected cells were cultured in high-glucose Dulbecco's modified Eagle's medium (DMEM), 10% fetal bovine serum (FBS), and 1% Antibiotic—Antimycotic (100×) in a 100-mm cell culture dish at 37° C. in a humidified 5% CO₂ incubator. PC-3 cells were cultured with Kaighn's Modification of Ham's F-12 Medium containing 10% fetal bovine serum, and 1% antibiotic and antimycotic n a 100-mm cell culture dish at 37° C. in a humidified 5% CO₂ incubator.

Construction of cDNA Plasmids Encoding Hybrid Enzymes.

COX linked PG synthase cDNAs of the hybrid enzyme COX-2 C-terminus was linked to the PGES N-terminus with 10 amino acids (COX-2-10aa-PGES) was constructed by a polymerase chain reaction (PCR) cloning approach. The resultant cDNA was sub-cloned into pcDNATM3.1 vectors containing a cytomegalovirus immediate-early promoter (11, 16).

### Establishing HEK293 Cells Line Expressing Recombinant Enzymes.

The recombinant enzyme was expressed in HEK293 cells. The cells were plated and transfected with the constructed pcDNA by the Lipofectamine 2000 method by following the manufacturer's instructions (Invitrogen) in a 6-well plate. The cells were harvested for assays approximately 48 hours after transfection. The enzyme expressions were determined by a Western blot analysis. A culture medium containing 12 μL/mL Geneticin (G418) was used to establish stable cell lines (11, 16).

### Cancer Cell PC-3 Migration Assay

One day before the experiment, about 8.4×106 of PC-3 cells were cultured in 6-well plates with 2 mL of growth medium. A marker line was drawn on the outside plates to identify viewing spots. The medium was pre-warmed, and each treatment (DMSO, NS-398, and #10) was mixed with the medium. The cells were washed with PBS and replaced with 1.5 mL of medium containing a treatment, and observed at 0, 15, 24, and 39-hour points. NIS-element AR 3.0 (Nikon Instruments Inc.) software was used to take images at different time points with 10× magnification, and to measure gap sizes.

### Determination of PGE₂ Inhibitory Effects Using HPLC-Scintillation Analyzer.

The effect of compounds that inhibited PGE₂ production were determined by HPLC. Different concentrations of the compounds (1 μM, 10 μM, 100 μM, and 1 mM) were added to HEK-COX-2-10aa-mPGES-1 cells for 10 minutes in a total volume of 225 μl (25 μl of the lysates+200 μl of 2.5 mM glutathione in PBS). Afterward, [¹⁴C]-AA which was purchased from Amersham Pharmacia Biotech (NJ, USA) was added as the substrate and incubated for 5 minutes. The reaction was then terminated by adding 200 μL of Buffer A. After centrifugation at 13000 rpm for 10 min, the supernatant was loaded onto a reverse phase C18 column (Varian Microsorb-MV 100-5, 4.6 mm×250 mm). Samples were run with the Buffer A (1.0 mL/min) with a gradient from 35 to 100% of acetonitrile for 40 min. The full metabolite profile was obtained by a flow scintillation analyzer (Packard 150TR, 16).

### Nuclear Magnetic Resonance (NMR) Spectroscopy.

¹H NMR spectra were recorded on a 600 MHz spectrometer at room temperature. ¹H NMR was measured in parts per million (ppm, δ) relative to the signal of tetramethylsilane (0.0 ppm). Data for ¹H NMR were reported as follows: chemical shift, multiplicity (s=singlet, d=doublet, t=triplet, q=quartet, m=multiplet, and br=broad), coupling constant J (Hz), and integration.

### Determination of the Molecular Weights of the Synthetic Compounds Using LC/MS

The synthetic compound was directly injected into the Waters Micromass LC/MS/MS system by an autosampler. The compounds were first separated by the HPLC C18 column (for positive mode) or polymer column (for negative mode [6]), and then automatically injected into the mass detector equipped with ESI or APCI source in a negative or positive mode.

### Results

Identification 4-(Fluoro-, Difluoro- or Trifluoro-Methyl)-Benzoic Acid-Modified Derivatives of 2,4-Diaminophenol Derivatives Inhibiting mPGES-1 by Docking Study Using High-Resolution Crystal 3D-Structure of the Native Trimer of Human mPGES-1.

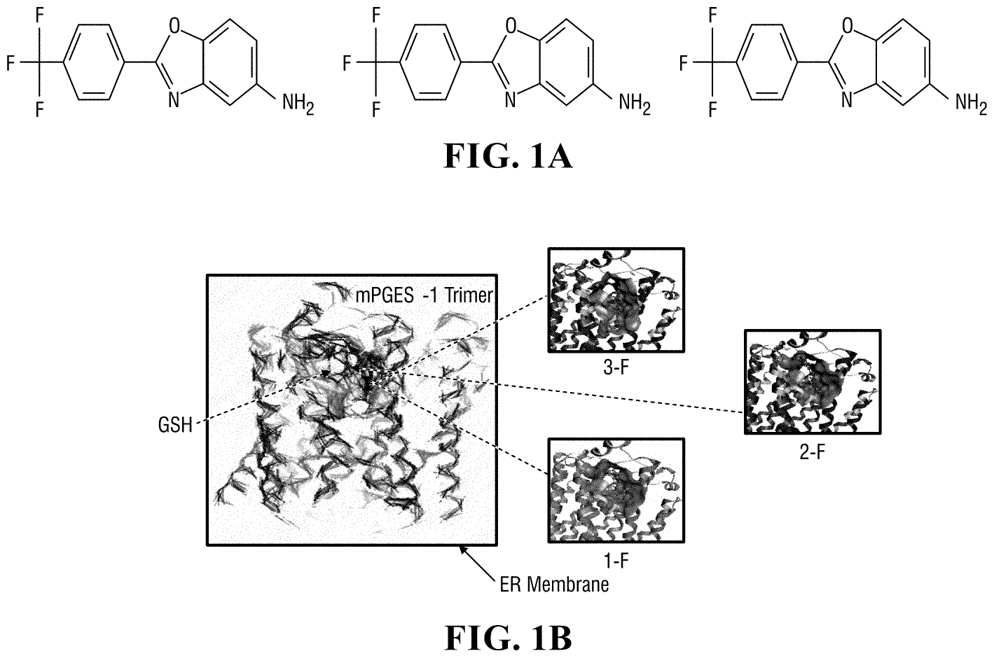

In a previous study, a 2,4-diaminophenol derivative was identified, compound 10, which bound to mPGES-1 specifically (11). Based on that observation other similar derivatives were searched for that could act as mPGES-1 inhibitors. To this end, a set of derivatives, the derivatives of 2,4-diaminophenol modified by 4-(fluoro-(1-F), difluoro-(2-F) or trifluoro-(3-F) methyl)-benzoic acid (FIG. 1A), were designed and docked into the substrate-binding pocket of a trimer form of the crystal structure of human mPGES-1 with high resolution using MOE software (FIG. 1B, left panel). The bound structures which interacted with the substrate-binding pocket of the mPGES-1 trimer were demonstrated individually (FIG. 1B right panels). The docking results for all three compounds with different scores are compared and summarized in Table 1. The binding affinities are 1F>2F>3F, but the scores are not significantly different. Thus, one of the compounds, the 3-F derivative, was selected to move on for chemical synthesis and biological activity characterization.

Identification 4-(Methyl-, Dimethyl- or Trimethyl)-Benzoic Acid-Modified Derivatives of 2,4-Diaminophenol Inhibiting mPGES-1 by Docking Study Using High-Resolution Crystal 3D-Structure of the Native Trimer of Human mPGES-1.

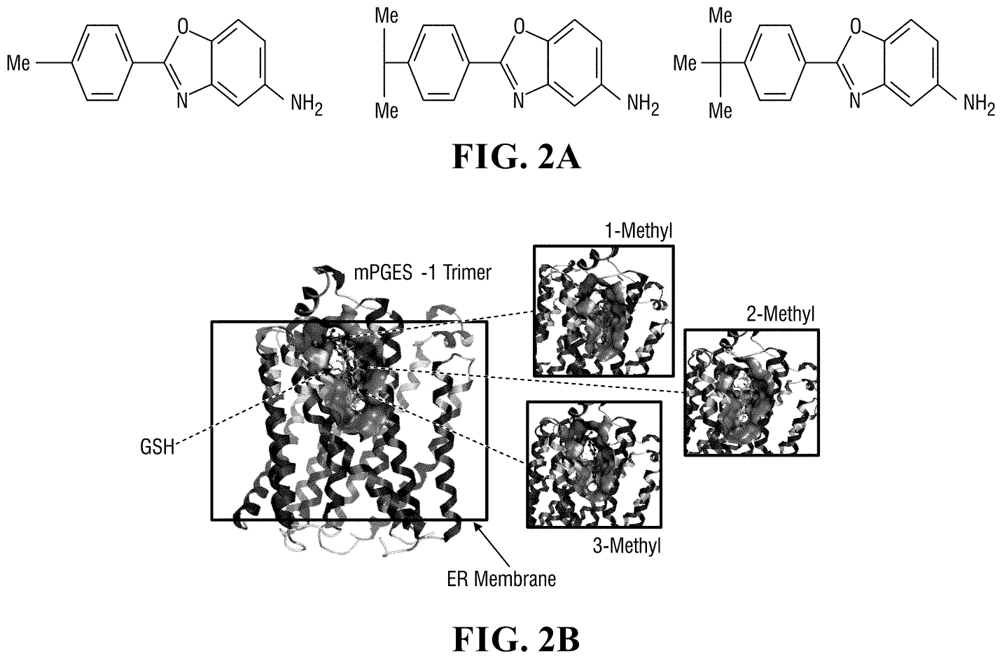

The second set of compounds, the 4-(methyl-(1-Me), dimethyl-(2-Me) or trimethyl (3-Me))-benzoic acid-modified derivatives of 2,4-diaminophenol (FIG. 2A) were designed and docked with the mPGES-1 trimer pocket. All of three Me derivatives binding to the mPGES-1 trimer pocket were identified (FIG. 2B left panel). The bound structures of the three Me derivatives which interacted with the substrate-binding pocket of the mPGES-1 trimer were also shown individually (FIG. 1B right panels). The docking results showed that the binding affinities to the mPGES-1 of the three Me derivatives are significantly higher than that of all of the three 1-F, 2-F, and 3-F derivatives. The detailed scores are compared and shown in Table 1. The binding affinities are ranked as 3-Me>2-Me>1-Me. The 3-Me derivative was selected for chemical synthesis and pharmacological tests. It was expected that both groups of the compounds, the —F and -Me derivatives, to have the potential to be excellent mPGES-1 inhibitors in experimental tests.

Docking of the Other Derivatives, 4-(Chloro-, Dichloro, or Trichloro-Methyl)-Benzoic Acid, and 4-(Phenyl, Diphenyl- or Tri-Phenol)-Benzoic Acid-Modified Derivatives of 2,4-Diaminophenol Inhibiting mPGES-1 by Docking Study Using High-Resolution Crystal 3D-Structure of the Native Trimer of Human mPGES-1.

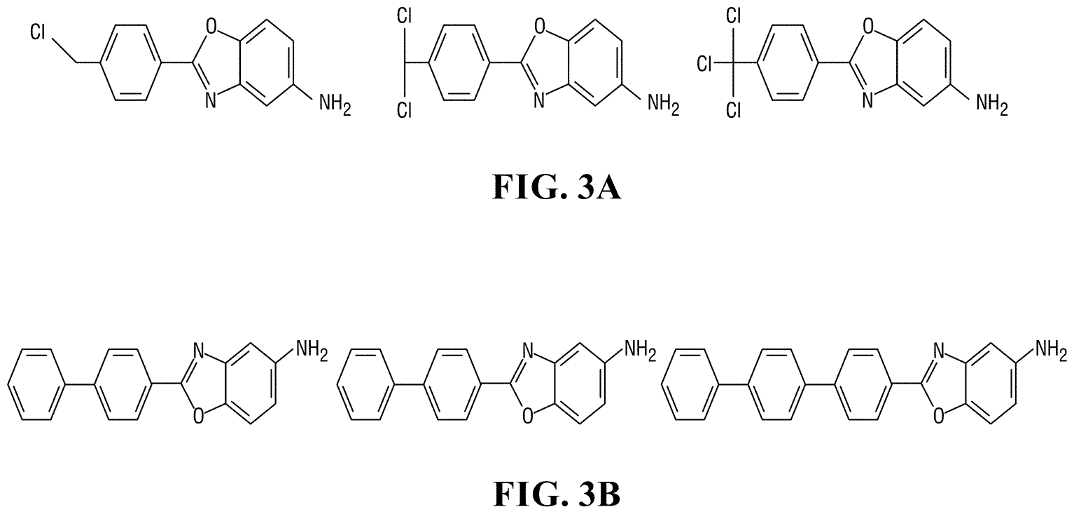

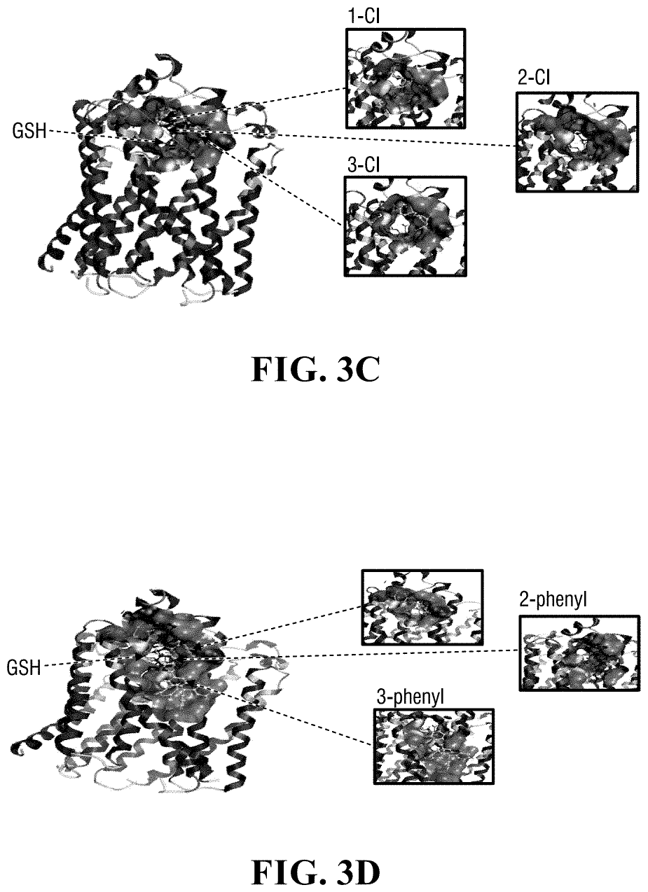

From the above studies, it was shown that the substitution of H with Methyl- and Flouro-groups at the positions shown in FIGS. 1A and 1B could change the binding affinities of the compounds. This demonstrated that chemical modifications to these positions are important to the optimized inhibition of mPGES-1. In order to find the best mPGES-1 inhibitor, a third set of compounds, 4-(chloro-(1-Cl), dichloro-(2-Cl), or trichloro(3-Cl)-methyl)-benzoic acid derivatives (FIG. 3A), and a fourth set of 4-(phenyl-(1-Phe), diphenyl-(2-Phe) or tri-phenol (3-Phe)-benzoic acid-modified derivatives of 2,4-diaminophenol designed and docked with the mPGES-1 pocket (FIG. 3). All Cl and Phe derivatives were identified as binding to the mGES-1 trimer pocket (FIG. 3). The docking results are compared and summarized in Table 1. The results indicate that the replacement of —Cl and -Phe could cause steric hindrance which limits binding to mPGES-1.

### Chemical Synthesis and Purification of the Trifluoro-Derivative as a Model for all of Fluoro-Derivatives

From the docking study, 1F, 2F, and 3F derivatives have similar binding affinities for the mPGES-1 pocket. Here, the chemical synthesis of the 3F derivative is described as an example. The method can be applied to the other 1F and 2F syntheses. The method for the synthesis of the 3F derivative was started from 2-amino, 4-nitrophenol, a low-cost compound. The chemical synthesis was performed by the addition of 4-(trifluoromethyl)benzoic acid to 2-amino, 4-nitrophenol in two-step reaction (FIG. 4A). TLC plates were used to monitor the reactions. The synthesized crude compound was simply purified by two steps: extraction and normal phase HPLC purification. The purification conditions and elution profile are summarized in FIG. 4B.

### Identification of the Structures of the Synthesized 3-F-2,4-Diaminophenol Derivative

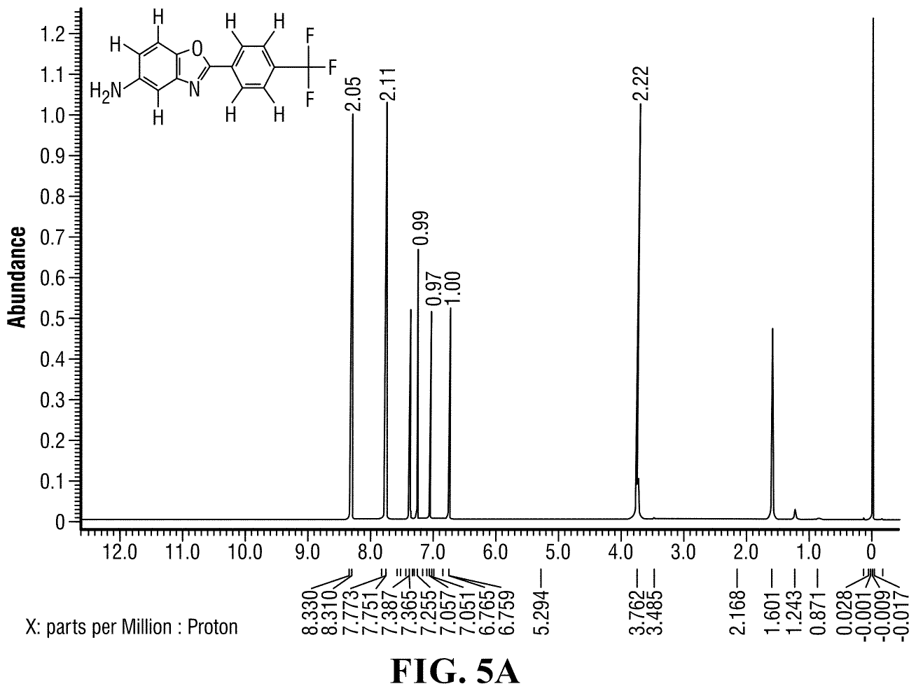

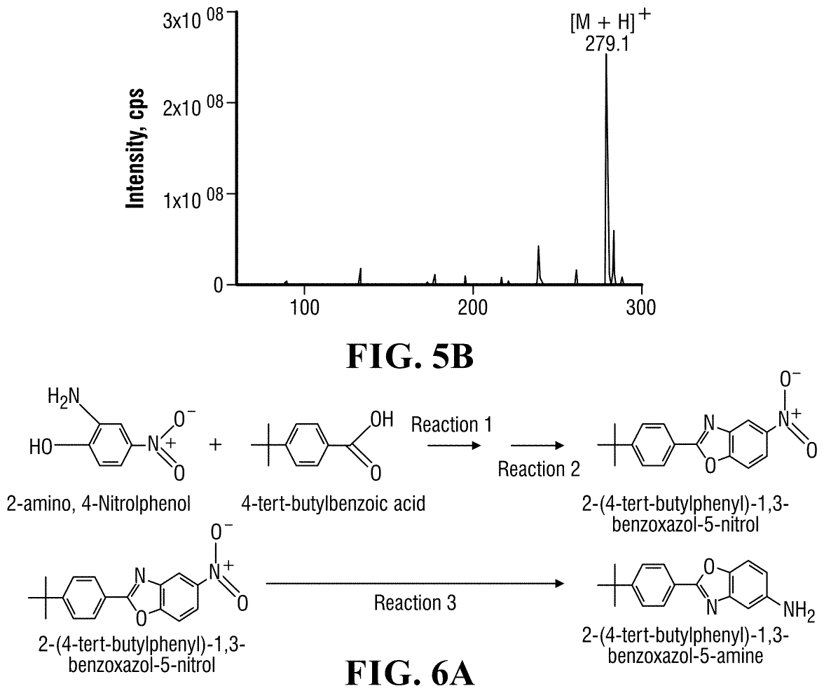

The structure of the 3-F derivative was confirmed by ¹H-NMR spectroscopy. The full assignments based on the 1D 1H NMR spectra are shown in FIG. 5A-B. The results have confirmed the chemical structures of the 3-F derivatives which are shown in FIG. 1 and FIG. 5A-B. One step further, the molecular weight for the synthesized compound, 2-[4-(trifluoromethyl)phenyl]-1,3-benzoxazole-5-amine was confirmed by LC/MS with a correct mass 279.1 (MH+).

### Chemical Synthesis and Structure Identification of 3-Me-2,4-Diaminophenol Derivative

The docking study adds substantially to ones understanding of 1Me, 2Me and 3Me derivatives which have very high binding-affinities to mPGES-1 within a similar range. 3-Me is used as an example for chemical synthesis. The chemical reaction for the 3Me synthesis was also from the 2-amino, 4-nitrophenol. The reaction steps are outlined in FIG. 6A. TLC analysis were also used to monitor those reactions. The synthesized crude 3F compound was purified by chemical extraction and HPLC purification. The structures of the 3Me derivative was also confirmed by 1D 1H-NMR spectroscopy. The products with the correct chemical structures were informed by the full assignments for the 1D 1H NMR spectroscopy (FIG. 6B). The molecular weights for the 3-M derivative was further confirmed by LC/MS with a correct mass 267.3 (MH+).

### Chemical Synthesis and Purification of 1-Cl and 1-Phe Derivatives

The docking study has a number of important implications for the finding that the Cl and Benzoic derivatives have low binding affinities to mPGES-1. But, these compounds can be used as negative controls and comparisons. On the other hand, these compounds could also be used as tools to test the docking accuracy. 1-Cl and 1-Phe (FIG. 7 and FIG. 8) were selected for chemical synthesis. The reaction steps are outlined in FIG. 7. TLC analysis plates were also used to monitor the reactions. The synthesized crude 1-Cl and 1-Phe derivatives have been purified by chemical extraction and normal phase HPLC purification as described above.

### Characterization of the 1-Cl and 2-Phenyl Derivatives by 1H NMR Spectroscopy.

Chloromethyl-benzoic acid modified derivative: 1H NMR: δ 4.61 (2H, s), 6.56 (1H, dd, J=8.4, 1.8 Hz), 7.40 (1H, dd, J=1.8, 0.5 Hz), 7.51-7.70 (3H, 7.57 (ddd, J=8.0, 1.3, 0.4 Hz), 7.63 (dd, J=8.4, 0.5 Hz)), 8.06 (2H, ddd, J=8.0, 1.6, 0.4 Hz).

Dichloromethyl-benzoic acid modified derivative: 1H NMR: δ 6.39 (1H, s), 6.57 (1H, dd, J=8.5, 1.8 Hz), 7.42 (1H, dd, J=1.8, 0.5 Hz), 7.58-7.76 (3H, 7.64 (dd, J=8.5, 0.5 Hz), 7.70 (ddd, J=8.1, 1.4, 0.4 Hz)), 8.05 (2H, ddd, J=8.1, 1.6, 0.4 Hz).

Trichloromethyl-benzoic acid modified derivative: 1H NMR: δ 6.61 (1H, dd, J=8.5, 1.9 Hz), 7.61 (1H, dd, J=1.9, 0.5 Hz), 7.69-7.85 (3H, 7.75 (ddd, J=8.1, 0.4 Hz), 7.79 (dd, J=8.5, 0.5 Hz)), 7.98 (2H, ddd, J=8.1, 1.7, 0.4 Hz).

Phenyl-benzoic acid modified derivative: 1H NMR: δ 3.94 (2H, s), 6.56 (1H, dd, J=8.4, 1.8 Hz), 7.11 (2H, dddd J=7.8, 1.3, 1.2, 05 Hz), 7.19-7.44 (4H. 7.25 tdd, J=7.8, 1.9, 0.5 Hz), 7.31 (tt, J=7.7, 1.3 Hz), 7.39 (dd, J=1.8, 0.5 Hz)), 7.49-7.69 (3H, 7.55 (ddd, J=8.0, 1.2, 0.4 Hz), 7.63 (dd, J=8.4, 0.5 Hz)), 7.94 (2H, ddd. J=8.0, 1.6, 0.4 Hz).

Diphenyl-benzoic acid modified derivative: 1H NMR: δ 5.59 (1H, s), 6.56 (1H, dd, J=8.4, 1.8 Hz), 7.11-7.45 (11H, 7.17 (dtd, J=7.9, 1.3, 0.5 Hz), 7.24 (tt, J=7.7 1 3 Hz), 7.32 (dddd, J=7.9, 7.7, 1.9, 0.5 Hz), 7.39 (dd, J=1.8, 0.5 Hz)), 7.57-7.69 (3H, 7.63 (ddd, J=8.2, 1.4, 0.4 Hz), 7.63 (dd, J=8.4, 0.5 Hz)), 8.03 (2H, ddd, J=8.2, 1.6, 0.4 Hz)

Tri-phenol-benzoic acid modified derivative: 1H NMR: δ 6.56 (1H, dd, J=8.4, 1.8 Hz), 7.16-7.34 (15H, 7.22 (dddd, J=7.9, 1.3, 1.2, 0.5 Hz), 7.23 (tt, J=7.7, 1.2 Hz), 7.26 (dddd, J=7.9, 7.7, 1.9, 0.5 Hz)), 7.40 (1H, dd, J=1.8, 0.5 Hz), 7.57-7.75 (3H, 7.63 (dd, J=8.4, 0.5 Hz), 7.68 (ddd, J=8.2, 1.5, 0.4 Hz)), 8.01 (2H, ddd, J=8.2, 1.7, 0.4 Hz).

Determination of the Biological Activity of the Synthesized Derivatives Targeting mPGES-1-Catalyzed PGE₂ Biosynthesis

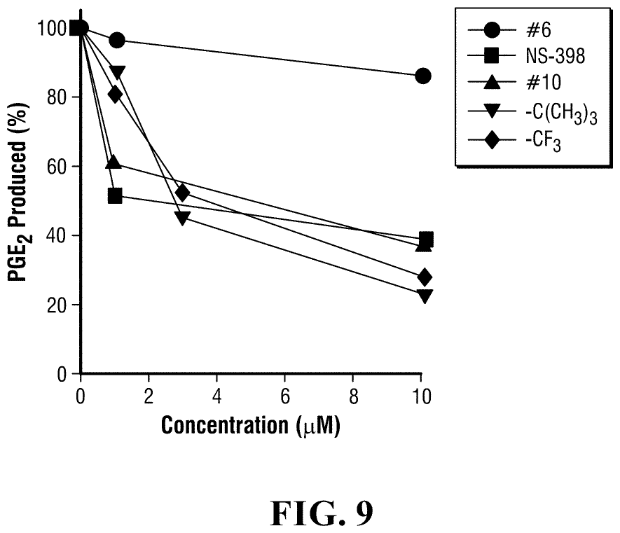

The inhibitory effect on PGE₂ production by mPGES-1 was determined by using previously developed HEK293 cells expressing our engineered Enzymelink, COX-2-10aa-mPGES-1. All of the three compounds, the 3-F, 2-Me, and 3-Me benzoic acids-modified 2,4-diaminophenol were able to inhibit inflammatory PGE₂ production with very similar dose-response concentrations of the COX-2 inhibitor (FIG. 9). This indicates that the backbone structure, 2,4-diaminophenol derivatives with the modifications at the position 1 —OH group and position 2 —NH2 group using benzoic acid is a key structure to generate the inhibitors targeting mPGES-1.

### Determination of Anti-Inflammatory Activity of the Derivatives Using Cancer-Cell Migration Assay.

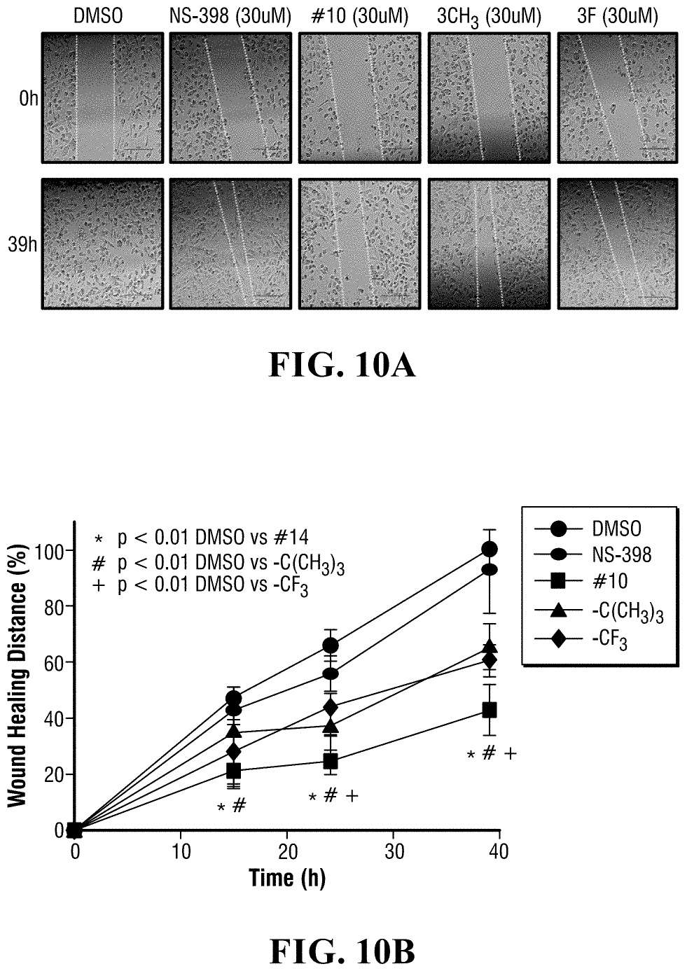

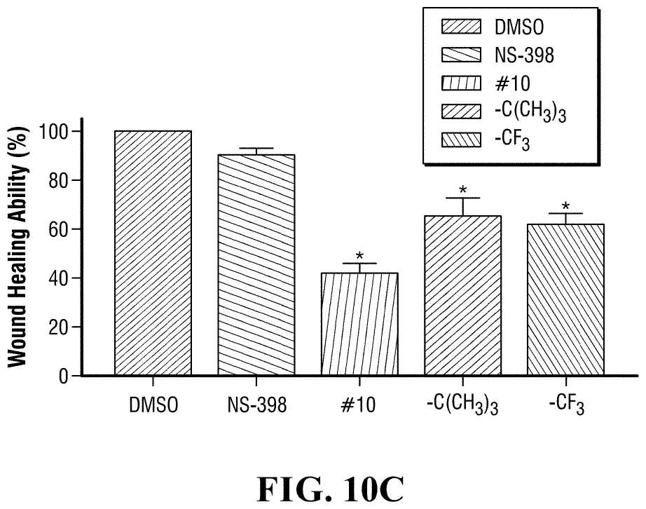

Inflammation is a factor that stimulates cell migration and is associated with a wide range of ailments including cancer (29). Inflammatory PGE₂ produced by mPGES-1 has been reported to be directly associated with cancer cell migration and development (30-33). Here, the cellular migration of the pancreatic cancer cell line, P-3 cells was used as a model to show the anti-inflammatory effects of the derivatives. A gap of the cultured P-3 cells was created first (FIG. 9, day 0). Then, the different concentrations of the derivatives or positive control, COX-2 inhibitor, were added to the cells. After 39 hours of culture, the gaps were measured (FIG. 10A) and quantified (FIG. 10B and FIG. 10C). All of the three derivatives, 2-Me, 3-Me, and 3-F benzoic acid-modified 2,4-diamoniphonel derivatives showed very effective inhibition of cancer cell migration within the gaps, similar to that of the positive control, COX-2 inhibitor, NS-398. DMSO was used as the negative control.

The side effects of COX-2 inhibitors, which include increased risk of heart disease (3-7), have limited their uses. Thus, there has been an increased focus on determining a replacement with similar anti-inflammatory effects as NSAIDs without the negative cardiovascular side effects. Since inducible mPGES-1 was identified as a key downstream enzyme that is coupled to inducible COX-2 (11-16) to produce the inflammatory PGE₂, it has become possible to replace COX-2 inhibitors with an mPGES-1 inhibitor, which is unlikely to have any negative impacts on patients with heart disease. However, an effective mPGES-1 inhibitor is not available in the market yet. The method to make 2,4-diaminophenol derivatives which specifically inhibit mPGES-1 as described herein provide a novel anti-inflammatory drug that targets mPGES-1.

The study described above has shown that the 1-3F and 1-3Me derivatives from 2,4-diaminophenol effectively bind to the pocket of mPGES-1, but not the COX-2 and PGIS. This finding provides a basis for the synthesis of compounds that exert their anti-inflammatory effects by reducing inflammatory PGE₂ synthesis using mPGES-1 inhibition. By targeting mPGES-1, COX-2, and PGIS, which are needed to produce the vascular protector prostacyclin, are not affected. The 3D structural models used in this method can be extended to other methods for use in the identification of other compounds which regulate prostanoid synthesis. This method has also indicated that other functional groups, such as the 1-3 Cl and phenyl group, may also impact the inhibition of mPGES-1.

For drug discovery, one of the key factors is that the potential drug can be synthesized easily at a low cost. The methods described here for synthesis of 2,4-diaminophenol derivatives are relatively simple and effective with high yields. The initial compound 2,4-diaminophenol is a popular and low-cost compound suitable for large-scale synthesis and cost effective production. The chemicals used for addition of the 1-3F and 1-3Me functional groups are also commercially available and easily obtained.

It should be indicated that other mPGEs-1 inhibitors have been tested (20,24,25). However, the derivative compounds disclosed herein are more specific to mPGES-1 and are more attractive candidates for the development of a new generation of anti-inflammatory drugs which can replace COX-2 inhibitors or be powerful alternatives which reduce the risk of heart disease risk for those who currently use the common COX-2 inhibitors, such as Celebrex.

There are several advantages of the current finding, which include: a) the chemical structures of the active compounds that specifically inhibit mPGES-1, but not COX and PGI₂ synthases, have been confirmed; b) due to the relatively simple chemical structures of the active compounds, they can be synthesized with less steps and low costs; c) the methods used to make the final active products of the 2,4-diaminophenol derivatives only require two-step reactions, which can be performed in any chemical lab; and d) due to only two-step reactions being involved, the purification and characterization steps are also minimized which saves production costs.

TABLE 1
Comparison of the scores for the docking of the derivatives
with the mPGES-1 trimer.

| Name | S Score | RMSD |
|---|---|---|
| 1-F | −4.9685 | 1.8743 |
| 2-F | −4.9529 | 1.4980 |
| 3-F | −4.7437 | 0.9538 |
| 1-Methyl | −5.0828 | 0.6300 |
| 2-Methyl | −5.1173 | 1.1271 |
| 3-Methyl | −5.3499 | 1.6791 |
| 1-Cl | 207.7725 | 3.6457 |
| 2-Cl | 1356.0842 | 2.9810 |
| 3-Cl | 315.2350 | 3.0853 |
| 1-phenyl | 741.1112 | 3.6780 |
| 2-phenyl | 480.7134 | 3.2397 |
| 3-phenyl | 82.2087 | 6.8950 |

### REFERENCES

1. Akasaka, Hironari, Shui-Ping So, and Ke-He Ruan. 2015. “Relationship of the Topological Distances and Activities between mPGES-1 and COX-2 versus COX-1: Implications of the Different Post-Translational Endoplasmic Reticulum Organizations of COX-1 and COX-2.” Biochemistry 54 (23): 3707-15. doi:10.1021/acs.biochem.5b00339.
2. Anti-inflammatory drugs in the 21st century. Rainsford K D. Subcell Biochem. 2007; 42:3-27. doi: 10.1007/1-4020-5688-5_1.PMID: 17612044 Review.
3. Selective COX-2 Inhibitors: A Review of Their Structure-Activity Relationships. Zarghi A, Arfaei S. Iran J Pharm Res. 2011 Fall; 10(4):655-83.PMID: 24250402.
4. Progress in COX-2 inhibitors: a journey so far. Chakraborti A K, Garg S K, Kumar R, Motiwala H F, Jadhavar P S. Curr Med Chem. 2010; 17(15):1563-93. doi: 10.2174/092986710790979980. PMID: 20166930 Review.
5. [Non-steroidal anti-inflammatory agents with selective inhibitory activity on cyclooxygenase-2. Interest and future prospects]. Blain H, Jouzeau J Y, Netter P, Jeandel C. Rev Med Interne. 2000 November; 21(11):978-88. doi: 10.1016/s0248-8663(00)00254-x. PMID: 11109595 Review. French.
6. Non-steroidal anti-inflammatory drug-induced cardiovascular adverse events: a meta-analysis. Gunter B R, Butler K A, Wallace R L, Smith S M, Harirforoosh S. J Clin Pharm Ther. 2017 February; 42(1):27-38. doi: 10.1111/jcpt.12484. Epub 2016 Dec. 26. PMID: 28019014 Review.
7. Vane, J R, Y S Bakhle, and R M Botting. 1998. “Cyclooxygenases 1 and 2.” Annual Review of Pharmacology and Toxicology 38 (January): 97-120. doi:10.1146/annurev.pharmtox.38.1.97.
8. Advance in understanding the biosynthesis of prostacyclin and thromboxane A2 in the endoplasmic reticulum membrane via the cyclooxygenase pathway. Ruan K H. Mini Rev Med Chem. 2004 August; 4(6):639-47. doi: 10.2174/1389557043403710. PMID: 15279598 Review.
9. Ruan, Ke-He, Hui Deng, and Shui Ping So. 2006. “Engineering of a Protein with Cyclooxygenase and Prostacyclin Synthase Activities That Converts Arachidonic Acid to Prostacyclin.” Biochemistry 45 (47): 14003-11. doi:10.1021/bi0614277.
10. Ruan, Ke-He He, Shui-Ping Ping So, Vanessa Cervantes, Hanjing Wu, Cori Wijaya, and Rebecca R. Jentzen. 2008. “An Active Triple-Catalytic Hybrid Enzyme Engineered by Linking Cyclo-Oxygenase Isoform-1 to Prostacyclin Synthase That Can Constantly Biosynthesize Prostacyclin, the Vascular Protector.” The FEBS Journal 275 (23): 5820-29. doi:10.1111/j.1742-4658.2008.06703.x.
11. Engineering ‘Enzymelink’ for screening lead compounds to inhibit mPGES-1 while maintaining prostacyclin synthase activity. Ruan D T, Tang N, Akasaka H, Lu R, Ruan K H. Future Med Chem. 2021 July; 13(13):1091-1103. doi: 10.4155/fmc-2021-0056. Epub 2021 Jun. 3. PMID: 34080888.
12. Makoto Murakami, Hiroaki Naraba, Toshihiro Tanioka, Natsuki Semmyo, Yoshihito Nakatan, Fumiaki Kojima, Tomomi Ikeda, Mai Fueki, Akinori Ueno,Sachiko Oh-ishi, and Ichiro Kudo. Regulation of Prostaglandin E2 Biosynthesis by Inducible Membrane-associated Prostaglandin E2 Synthase That Acts in Concert with Cyclooxygenase-2, J. BIOL. CHEM. Vol. 275, No. 42, Issue of October 20, pp. 32783-32792, 2000
13. Nakanishi, Masako, David C. Montrose, Patsy Clark, Prashant R. Nambiar, Glenn S. Belinsky, Kevin P. Claffey, Daigen Xu, and Daniel W. Rosenberg. 2008. “Genetic Deletion of mPGES-1 Suppresses Intestinal Tumorigenesis.” Cancer Research 68 (9): 3251-59. doi:10.1158/0008-5472.CAN-07-6100.
14. Nakanishi, Masako, Vijay Gokhale, Emmanuelle J Meuillet, and Daniel W Rosenberg. 2010. “MPGES-1 as a Target for Cancer Suppression. A Comprehensive Invited review ‘Phospholipase A2 and Lipid Mediators.’ Biochimie. Elsevier Masson SAS. doi:10.1016/j.biochi.2010.02.006.
15. Kamei, Daisuke, Makoto Murakami, Yoshihito Nakatani, Yukio Ishikawa, Toshiharu Ishii, and Ichiro Kudo. 2003. “Potential Role of Microsomal Prostaglandin E Synthase-1 in Tumorigenesis.” Journal of Biological Chemistry 278 (21): 19396-405. doi:10.1074/jbc.M213290200.
16. Ruan, Ke-He, Vanessa Cervantes, and Shui Ping So. 2009. “Engineering of a Novel Hybrid Enzyme: An Anti-Inflammatory Drug Target with Triple Catalytic Activities Directly Converting Arachidonic Acid into the Inflammatory Prostaglandin E2.” Protein Engineering, Design and Selection 22 (12): 733-40. doi:10.1093/protein/gzp058.
17. Prostaglandin E synthase. Murakami M, Nakatani Y, Tanioka T, Kudo I. Prostaglandins Other Lipid Mediat. 2002 August; 68-69:383-99. doi: 10.1016/s0090-6980(02)00043-6. PMID: 12432931 Review.
18. Microsomal prostaglandin E synthase (mPGES)-1, mPGES-2 and cytosolic PGES expression in human gastritis and gastric ulcer tissue. Gudis K, Tatsuguchi A, Wada K, Futagami S, Nagata K, Hiratsuka T, Shinji Y, Miyake K, Tsukui T, Fukuda Y, Sakamoto C. Lab Invest. 2005 February; 85(2):225-36. doi: 10.1038/labinvest.3700200. PMID: 15531909.
19. The terminal prostaglandin synthases mPGES-1, mPGES-2, and cPGES are all overexpressed in human gliomas. Mattila S, Tuominen H, Koivukangas J, Stenback F. Neuropathology. 2009 April; 29(2):156-65. doi: 10.1111/j.1440-1789.2008.00963.x. MID: 19347995.
20. Membrane prostaglandin E synthase-1: a novel therapeutic target. Samuelsson B, Morgenstern R, Jakobsson P J. Pharmacol Rev. 2007 September; 59(3):207-24. doi: 10.1124/pr.59.3.1. PMID: 17878511 Review.
21. Cardiovascular effects of cyclooxygenase-2 inhibitors: a mechanistic and clinical perspective. Patrono C. Br J Clin Pharmacol. 2016 October; 82(4):957-64. doi: 10.1111/bcp.13048. Epub 2016 Jul. 18. PMID: 27317138 Free PMC article. Review.
22. The cardiovascular pharmacology of COX-2 inhibition. Fries S, Grosser T. Hematology Am Soc Hematol Educ Program. 2005:445-51. doi: 10.1182/asheducation-2005.1.445. PMID: 16304418
23. Structure-based discovery of mPGES-1 inhibitors suitable for preclinical testing in wild-type mice as a new generation of anti-inflammatory drugs. Kai Ding, Ziyuan Zhou, Shurong Hou, Yaxia Yuan, Shuo Zhou, Xirong Zheng, Jianzhong Chen, Charles Loftin, Fang Zheng, Chang-Guo Zhan Sci Rep. 2018; 8: 5205. Published online 2018 Mar. 26. doi: 10.1038/s41598-018-23482-4 PMCID: PMC5979965
24. Discovery of 3-hydroxy-3-pyrrolin-2-one-based mPGES-1 inhibitors using a multi-step virtual screening protocol Gianluigi Lauro, Vincenza Cantone, Marianna Potenza, Katrin Fischer, Andreas Koeberle, Oliver Werz, Raffaele Riccio, Giuseppe Bifulco Medchemcomm. 2018 Dec. 1; 9(12): 2028-2036. Published online 2018 Nov. 20. doi: 10.1039/c8md00497h PMCID: PMC6336085
25. DREAM-in-CDM Approach and Identification of a New Generation of Anti-inflammatory Drugs Targeting mPGES-1 Shuo Zhou, Ziyuan Zhou, Kai Ding, Yaxia Yuan, Charles Loftin, Fang Zheng, Chang-Guo Zhan Sci Rep. 2020; 10: 10187. Published online 2020 Jun. 23. doi: 10.1038/s41598-020-67283-0 PMCID: PMC7311425
26. Chancellor D R, Davies K E, De Moor O, Dorgan C R, Johnson P D, Lambert A G, Lawrence D, Lecci C, Maillol C, Middleton P J, Nugent G, Poignant S D, Potter A C, Price P D, Pye R J, Storer R, Tinsley J M, van Well R, Vickers R, Vile J, Wilkes F J, Wilson F X, Wren S P, Wynne G M. Discovery of 2-arylbenzoxazoles as upregulators of utrophin production for the treatment of Duchenne muscular dystrophy. J Med Chem. 2011 May 12; 54(9):3241-50. doi: 10.1021/jm200135z. Epub 2011 Apr. 15. PMID: 21456623.
27. Karatas E, Foto E, Ertan-Bolelli T, Yalcin-Ozkat G, Yilmaz S, Ataei S, Zilifdar F, Yildiz I. Discovery of 5-(or 6)-benzoxazoles and oxazolo[4,5-b]pyridines as novel candidate antitumor agents targeting hTopo IIα. Bioorg Chem. 2021 July; 112:104913. doi: 10.1016/j.bioorg.2021.104913. Epub 2021 Apr. 14. PMID: 33945950.)
28. Luz J G, Antonysamy S, Kuklish S L, Condon B, Lee M R, Allison D, Yu X P, Chandrasekhar S, Backer R, Zhang A, Russell M, Chang S S, Harvey A, Sloan A V, Fisher M J. Crystal Structures of mPGES-1 Inhibitor Complexes Form a Basis for the Rational Design of Potent Analgesic and Anti-Inflammatory Therapeutics. J Med Chem. 2015 Jun. 11; 58(11):4727-37. doi: 10.1021/acs.jmedchem.5b00330. Epub 2015 May 20. PMID: 25961169.
29. Liggett, Jason L, Xiaobo Zhang, Thomas E Eling, and Seung Joon Baek. 2014. “Anti-Tumor Activity of Non-Steroidal Anti-Inflammatory Drugs: Cyclooxygenase-Independent Targets.” Cancer Letters 346 (2). Elsevier Ireland Ltd: 217-24. doi:10.1016/j.canlet.2014.01.021.
30. Nakanishi, Masako, David C. Montrose, Patsy Clark, Prashant R. Nambiar, Glenn S. Belinsky, Kevin P. Claffey, Daigen Xu, and Daniel W. Rosenberg. 2008. “Genetic Deletion of mPGES-1 Suppresses Intestinal Tumorigenesis.” Cancer Research 68 (9): 3251-59. doi:10.1158/0008-5472.CAN-07-6100.
31. Kamei, Daisuke, Makoto Murakami, Yoshihito Nakatani, Yukio Ishikawa, Toshiharu Ishii, and Ichiro Kudo. 2003. “Potential Role of Microsomal Prostaglandin E Synthase-1 in Tumorigenesis.” Journal of Biological Chemistry 278 (21): 19396-405. doi:10.1074/jbc.M213290200.
32. Nakanishi, Masako, Vijay Gokhale, Emmanuelle J Meuillet, and Daniel W Rosenberg. 2010. “MPGES-1 as a Target for Cancer Suppression. A Comprehensive Invited review ‘Phospholipase A₂ and Lipid Mediators.’ Biochimie. Elsevier Masson SAS. doi:10.1016/j.biochi.2010.02.006.
33. Ricciotti, Emanuela, and Garret a FitzGerald. 2011. “Prostaglandins and Inflammation.” Arteriosclerosis, Thrombosis, and Vascular Biology 31 (5): 986-1000. doi:10.1161/ATVBAHA.110.207449.

What is claimed:

1. The modification of the position 1, —OH group (R1) and position 2 —NH group (R2) of 2-amino, 4-nitrol phenol for producing a compound of Formula I having the properties of anti-inflammatory activity, inhibiting PGE₂ biosynthesis and activity, and mPGES-1 activity:

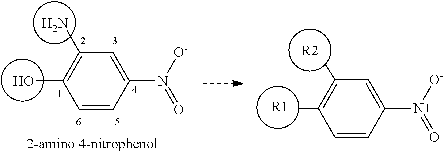

2. The modification of the position 1, —OH group (R1) and position 2 —NH group (R2) of 2,4-diaminophenol for producing a compound of Formula II having the properties of anti-inflammatory activity, inhibiting PGE₂ biosynthesis and activity, and mPGES-1 activity:

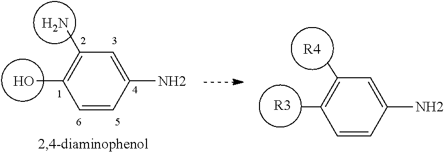

3. Compounds according to claim 1, where R1 and R2 are modified by benzoic acid resulting in a compound of Formula III, said compound having the properties of anti-inflammatory activity, inhibiting PGE₂ biosynthesis and activity, and mPGES-1 activity:

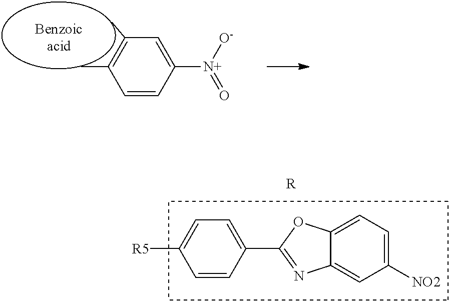

4. Compounds according to claim 2, where R3 and R4 are modified by benzoic acid resulting in the compound of formula (IV) said compound having the properties of anti-inflammatory activity, inhibiting PGE2 biosynthesis and activity, and mPGES-1 activity:

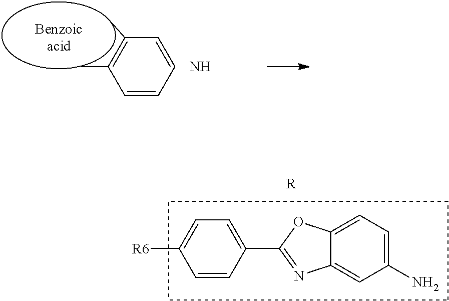

5. The compound according to claim 3, wherein:
(i) R5 is a fluoro-, difluoro- or trifluoro-group said compound having the properties of anti-inflammatory activity, inhibiting PGE₂ biosynthesis and activity, and mPGES-1 activity: 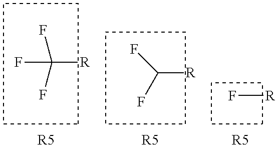
(ii) R5 is a methyl-, dimethyl- or trimethyl-group said compound having the properties of anti-inflammatory activity, inhibiting PGE₂ biosynthesis and activity, and mPGES-1 activity: 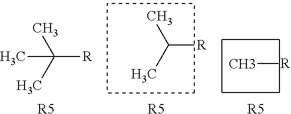
(iii) R5 is a chloro-, dichloro, or trichloro-group said compound having the properties of anti-inflammatory activity, inhibiting PGE₂ biosynthesis and activity, and mPGES-1 activity: 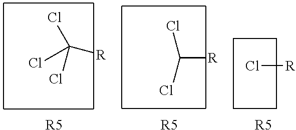
or (iv) R5 is, phenyl, diphenyl- or triphenyl-group said compound having the properties of anti-inflammatory activity, inhibiting PGE₂ biosynthesis and activity, and mPGES-1 activity:

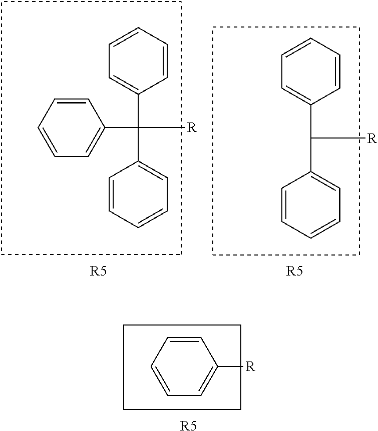

6. The compound according to claim 4, wherein;
(i) R6 is a fluoro-, difluoro- or trifluoro-group said compound having the properties of anti-inflammatory activity, inhibiting PGE₂ biosynthesis and activity, and mPGES-1 activity: 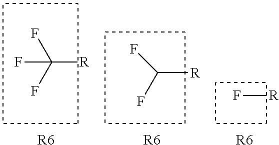
(ii) R6 is a methyl-, dimethyl- or trimethyl-group said compound having the properties of anti-inflammatory activity, inhibiting PGE₂ biosynthesis and activity, and mPGES-1 activity: 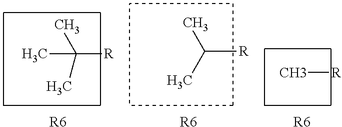
(iii) R6 is a chloro-, dichloro-, or trichloro-group said compound having the properties of anti-inflammatory activity, inhibiting PGE₂ biosynthesis and mPGES-1 activity: 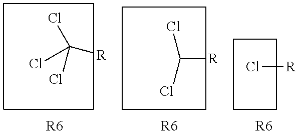
or (iv) R6 is a phenyl, diphenyl- or triphenyl-group said compound having the properties of anti-inflammatory activity, inhibiting PGE₂ biosynthesis and activity, and mPGES-1 activity:

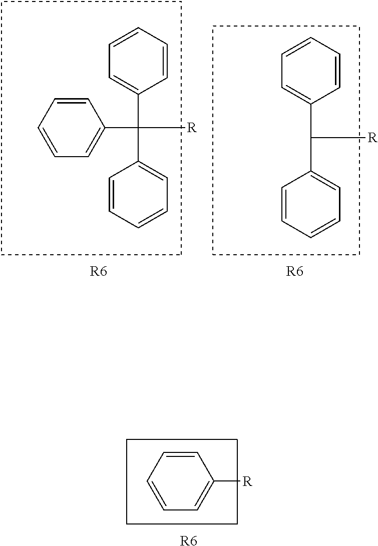

7. A pharmaceutical composition comprising one or more of the compounds or derivatives of claim 5 in a pharmaceutically acceptable form.

8. A pharmaceutical composition comprising one or more of the compounds or derivatives of claim 6 in a pharmaceutically acceptable form.

9. The compounds or derivatives of claim 5 for use in inhibition of PGE₂ biosynthesis and activity.

10. The compounds or derivatives of claim 6 for use in inhibition of PGE₂ biosynthesis and activity.

11. The pharmaceutical composition of claim 7 for use in inhibition of PGE₂ biosynthesis and activity, in vitro or in vivo.

12. The pharmaceutical composition of claim 8 for use in inhibition of PGE₂ biosynthesis and activity, in vitro or in vivo.

13. The compounds or derivatives of claim 5, for use in inhibition of mPGES-1 activity.

14. The compounds or derivatives of claim 6, for use in inhibition of mPGES-1 activity.

15. The pharmaceutical composition of claim 7, for use in inhibition of mPGES-1 activity, in vitro or in vivo.

16. The pharmaceutical composition of claim 8, for use in inhibition of mPGES-1 activity, in vitro or in vivo.

17. The pharmaceutical composition of claim 7, for use in treatment of inflammation, inflammatory diseases or disorders, pain, fever, arthritis, cancer, vascular, inflammation, neuronal inflammation, and heart disease. in a subject.

18. The pharmaceutical composition of claim 8, for use in treatment of inflammation, inflammatory diseases or disorders, pain, fever, arthritis, cancer, vascular, inflammation, neuronal inflammation, and heart disease. in a subject.

19. The compounds or derivatives of claim 5, for prevention and treatment of a disease related to mPEGES-1 enzyme biosynthesis and/or activity.

20. The compounds or derivatives of claim 6, for prevention and treatment of a disease related PEG₂ biosynthesis and/or activity.
